(19) United States
(12) Patent Application Publication
(10) Pub. No.: US 2026/0251629 A1
(43) Pub. Date: Aug. 27, 2026

(54) DEVICES, SYSTEMS, AND METHODS FOR NON-CONTACT ALLERGEN DETECTION

(71) Applicant: Samvith Mahadevan, Austin, TX (US)

(72) Inventor: Samvith Mahadevan, Austin, TX (US)

(21) Appl. No.: 19/060,561

(22) Filed: Feb. 21, 2025

(51) Int. Cl. G01N 33/00; G01N 33/02

(52) U.S. Cl. CPC G01N 33/0027; G01N 33/02

(57) ABSTRACT

An allergen detection device, system, and method for detecting the presence of allergens is disclosed. The allergen detection device includes an olfactory sensor array arranged to be exposed to gases emitted from a food sample or an environmental sample. Heating elements heat the olfactory sensor array to one or more specified temperatures. A voltage measurement device measures a voltage signal and associate the voltage signal with a presence of one or more allergens in the food sample or the environmental sample.

### TECHNICAL FIELD

The embodiments disclosed herein generally relate allergen detection devices, specifically to a non-contact smells sensor system capable of detecting and classifying allergens in food samples. The invention further applies to detecting mold, airborne allergens, and other contaminants.

### BACKGROUND

Food allergen detection is a critical concern for individuals with allergies. Current methods involve dip or surface tests requiring direct food contact, which may introduce hygiene concerns and risk of contamination. Other methods, such as gas chromatography and ELISA tests, require laboratory analysis and are time-consuming. Existing market solutions often detect only one or two allergens and are costly.

### SUMMARY OF THE INVENTION

This summary is provided to introduce a variety of concepts in a simplified form that is further disclosed in the detailed description of the embodiments. This summary is not intended for determining the scope of the claimed subject matter.

The embodiments provided herein relate to an allergen detection device, system, and method for detecting the presence of allergens is disclosed. The allergen detection device includes an olfactory sensor array arranged to be exposed to gases emitted from a food sample or an environmental sample. Heating elements heat the olfactory sensor array to one or more specified temperatures. A voltage measurement device measures a voltage signal and associate the voltage signal with a presence of one or more allergens in the food sample or the environmental sample.

The disclosed invention addresses the limitations of prior-existing systems by introducing a non-contact, olfactory-based sensor system that detects a wide range of allergens in food samples at varying temperatures with high accuracy and a fast response time.

The system provides a means for the non-contact detection of allergens, eliminating the need to have physical contact with food (e.g., chemical-based test strips, fluorescence PCR, or molecular binding assays), The disclosed embodiments detects allergens through airborne food vapors. This feature eliminates the risk of cross-contamination, making it more hygienic and practical for users.

The system provides temperature-independent detection of the allergens using heated sensors. Previous technologies have required users to prepare food in a specific manner and the food must be at a specific temperature for an accurate measurement. The disclosed embodiments utilize the heating elements to heat the sensors to an appropriate temperature, allowing the sensor array to detect allergens in both hot and cold foods, ensuring consistent performance across different food states.

While existing solutions typically detect one or two specific allergens at a time (e.g., almond detection using fluorescence PCR), the disclosed embodiments enable simultaneous multi-allergen detection by leveraging an olfactory sensor array and pattern recognition.

The disclosed system uses convolutional neural networks (CNNs) and ADAM optimization to improve allergen detection accuracy, while the existing solutions rely on pre-defined allergen detection methods (e.g., molecular binding, fluorescence, or antibody-based tests). The continuous learning model allows future updates to enhance allergen classification without modifying hardware.

The allergen detection device is provided having a portable and consumer-friendly design which can be integrated into consumer-friendly devices such as a standalone sensor, phone case, or smart home device. This eliminates the need for traditional laboratory-based testing of a sample which often require hours to complete (such as in the examples of PCR, IgG antibody test kits, ELISA, fluorescence PCR, etc.). The embodiments provided herein are capable of testing a sample within 1-3 minutes, providing fast and accurate results to the user. Using this device, there is no need to prepare samples for the assay as the sample is food vapors or airborne particles in the environment.

The system may be in operable communication with auxiliary devices (e.g., a laptop, smartphone, etc.) via a network to transmit the results. This allows the user to remotely receive results of the test to determine if allergens are present in the food sample or in the environment.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1A illustrates a perspective view of the allergen detection device illustrating the PCB board with an olfactory sensor array, according to some embodiments;

FIG. 1B illustrates an elevation view of the bottom side of the allergen detection device and PCB board, according to some embodiments;

FIG. 2 illustrates a perspective view of the sensor housing clamp designed to secure the sensor array in position, according to some embodiments;

FIG. 3 illustrates an elevation view of the sensor mount capable of being attached to the clamp, according to some embodiments;

FIG. 4 illustrates an elevation view of a smartphone case having a mounting system to releasably engage with the allergen detection device, according to some embodiments;

FIG. 5 illustrates a flow diagram of the process for developing the allergen detection system, including sensor selection, data collection, machine learning model training, simulated testing, and real-world validation, according to some embodiments;

FIG. 6 illustrates a flow diagram of the model training process to improve allergen detection accuracy, according to some embodiments; and

FIG. 7 illustrates a schematic of the working principle of the allergen detection system while drawing an analogy between human olfaction and machine-based allergen detection, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing in detail exemplary embodiments, it is noted that the embodiments reside primarily in combinations of components related to particular devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

In general, the embodiments provided herein relate to a device, system, and method for detecting the presence of allergens in a food sample or in an environment. The device is capable of non-contact detection using an olfactory-based sensor array that detects a wide range of allergens in food and environmental samples.

The system includes a sensors array comprised of multiple metal oxide sensors capable of detecting specific allergen signatures. Each sensor operates at different heating profiles to optimize detection. The sensor array includes heating elements that allow controlled heating. The system dynamically adjusts heating profiles to maximize sensitivity across different food temperatures. A microcontroller processes raw voltage signals from the sensor array and feeds data into the machine learning model for classification. Data is transmitted to a mobile application via Bluetooth or Wi-Fi for real-time result display. The system may be powered through a rechargeable battery or external power source, allowing portability and providing convenience.

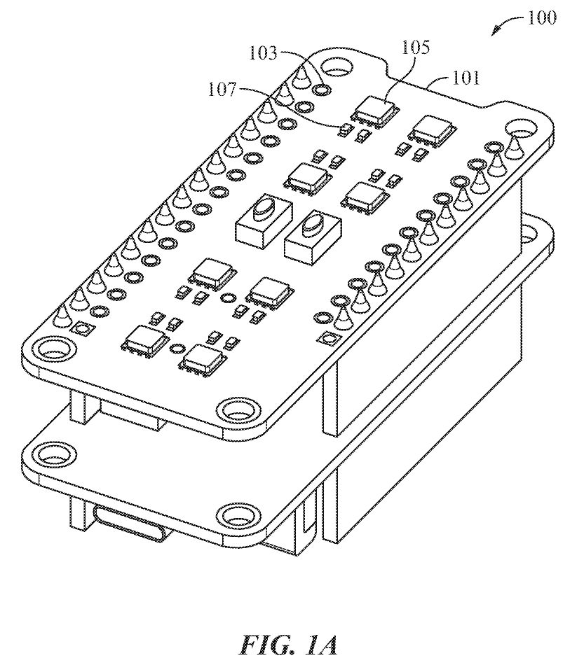

FIG. 1A and 1B illustrate the allergen detection device 100 illustrating the PCB board 101 with an olfactory sensor array 103. The PCB board 101 integrates metal oxide olfactory sensors and environmental monitoring capabilities to enhance allergen detection accuracy. The PCB board 101 houses an olfactory sensor array 103 that detects airborne allergenic compounds emitted from food samples. These sensors operate based on changes in electrical conductivity when exposed to specific allergen-associated volatile organic compounds (VOCs). The olfactory sensor array 103 may be comprised of metal oxide sensors 105. The PCB board 101 also incorporates temperature, humidity, and pressure sensors to adjust detection parameters dynamically. Since environmental conditions such as temperature and humidity can influence gas molecule dispersion and sensor response, the PCB Board 101 provides real-time environmental compensation to improve detection accuracy. The PCB board 101 also includes a microcontroller interface for real-time data processing and communication with external devices. It is designed to be compact, requires minimal energy to function, and is suitable for integration into portable allergen detection systems, including handheld devices or smartphone-mounted configurations. Switches 109 are positioned on the PCB board 101 to allow the user to select operations controls of the device.

In further reference to FIG. 1A and FIG. 1B, the PCB board 101 is electrically connected to the heating elements 107 that adjust the sensor temperatures to optimize allergen detection across hot and cold food samples and to accurately classify the allergen(s). Additionally, the PCB board 101 supports wireless data transmission via Bluetooth or Wi-Fi, allowing seamless connectivity with a mobile application for real-time allergen classification and result visualization. The PCB Board 101 works in conjunction with a machine learning algorithm deployed on the device’s microcontroller or cloud-based system, enabling it to continuously refine allergen detection and adapt to new allergens through remote software updates.

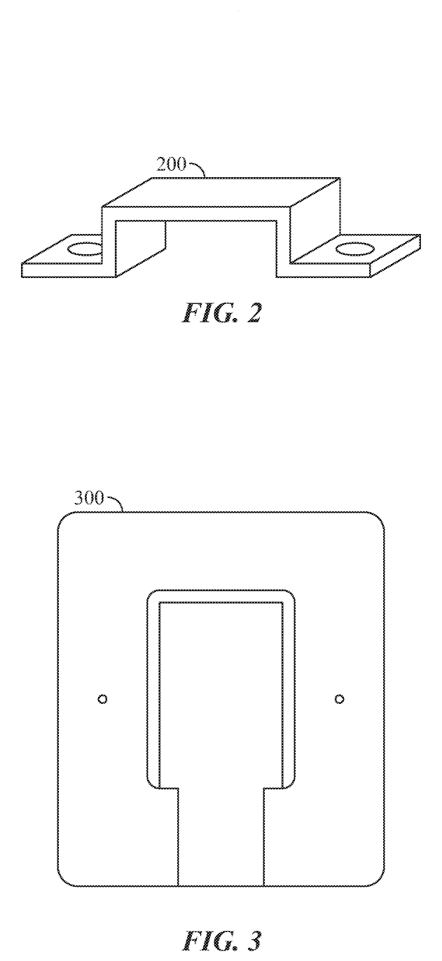

In reference to FIG. 1B, the backside 150 of the PCB board 101 includes a battery slot 155 capable of at least partially retaining a battery of the PCB board 101. An SD card holder 157 is provided to receive and store data captured by the device 100. Receivers 111 are capable of slidingly engaging with the mount (see FIG. 3) or other auxiliary devices having a complimentary mount.

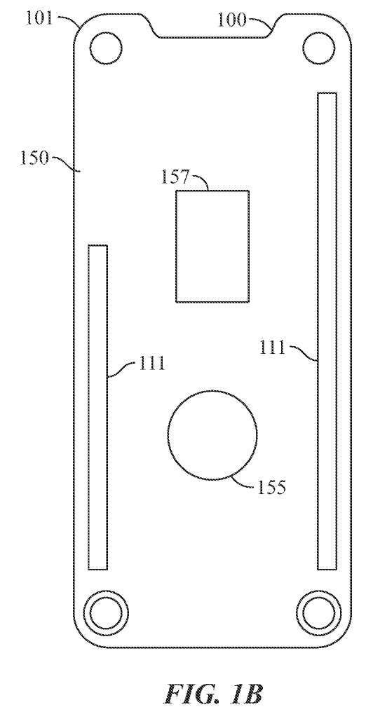

FIG. 2 illustrates a perspective view of the sensor housing clamp 200 designed to secure the sensor array in position a structural component designed to securely hold the olfactory sensor array in place while ensuring optimal exposure to airborne allergenic compounds. The clamp 200 is configured to maintain a precise sensor position over the food sample, preventing unintended movement that could impact detection accuracy. The clamp 200 may also prevent accidental contact with the food and avoid cross-contamination risks. The housing helps maintain a seal with the food container being tested to enhance the metal oxide sensor’s reaction with the food vapors. The clamp also includes a gap in the sensor opening, allowing allergenic gas molecules to flow freely towards the metal oxide sensors without obstruction. The housing clamp is designed to be modular and adaptable, enabling its integration into various mounting configurations, such as a phone case, standalone device, or auxiliary attachment. Additionally, the clamp features adjustable fasteners that allow users to modify the sensor’s height and orientation relative to the sample, enhancing detection sensitivity across different food environments. The clamp 200 helps maintain consistent and reliable allergen detection, making the system effective in both controlled laboratory settings and real-world consumer

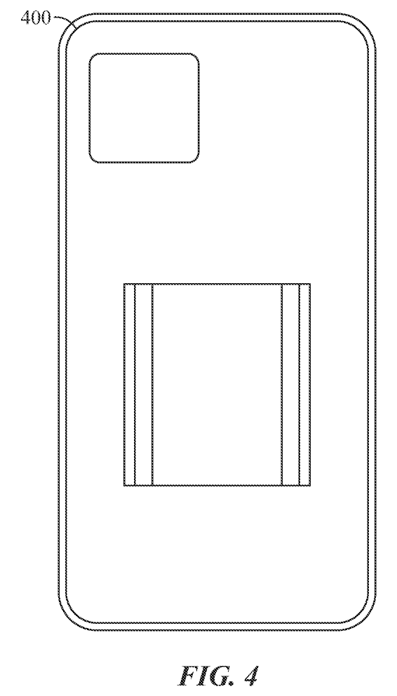

FIG. 3 illustrates a mounting system 300 capable of releasably engaging to a smartphone case 400 (see FIG. 4). The mounting system 300 and smartphone case 400 provide an integrated platform allowing the device 100 to be mounted to a smartphone (or similar device) to enable a portable, user-friendly, and efficient device capable of being deployed at any time.

In some embodiments, the smartphone case 400 provides a dedicated cavity for the battery, sensor array, and/or other components to ensure the reception allergenic vapors toward the metal oxide sensors. The mounting system 300 further enhances versatility by enabling adjustable positioning of the sensor relative to the food sample, ensuring optimal gas exposure for reliable detection. The smartphone-integrated design allows users to view real-time allergen detection results via a mobile application, which receives data from the sensor module via Bluetooth or Wi-Fi connectivity. Additionally, the case and mount design support detachable configurations, allowing users to remove or replace the sensor module as needed. This system transforms a standard smartphone into a portable allergen detection device, making it practical for everyday use in restaurants, grocery stores, and home kitchens, while maintaining a compact and aesthetically unobtrusive form factor.

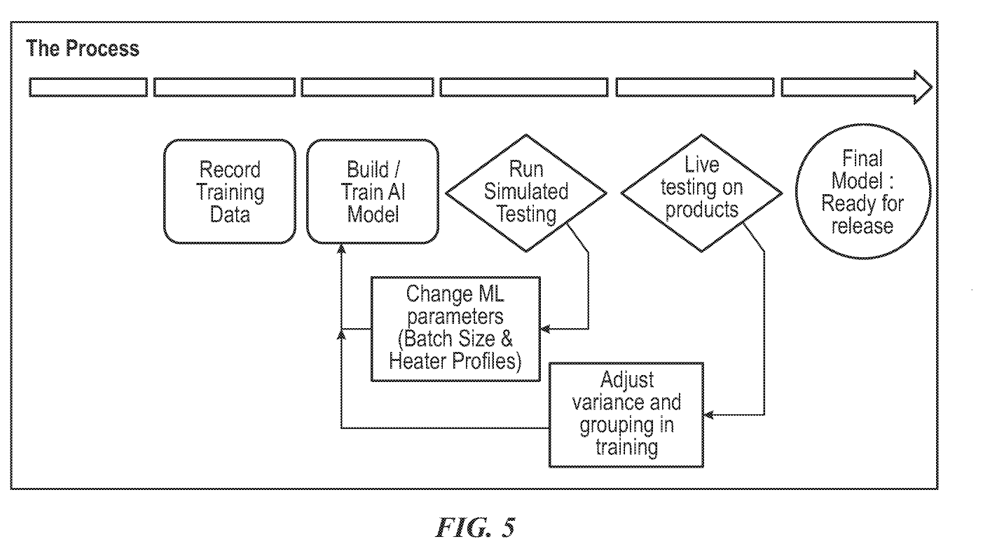

FIG. 5 illustrates a flow diagram of the process for developing the allergen detection system, including sensor selection, data collection, machine learning model training, simulated testing, and real-world validation. The metal oxide sensors are capable of being calibrated for specific allergen detection. Data from various allergens is collected and recorded to establish a baseline for detection. This involves gathering voltage response signals from allergen exposure at different sensor heating profiles. A machine learning model is developed using the training data, focusing on key allergens. Training is conducted using a convolutional neural network (CNN) with ADAM optimization. The model undergoes simulated testing to evaluate its performance under different conditions. Adjustments are made to improve sensitivity and specificity. Once complete, real-world food samples are tested to fine-tune accuracy. The model is further refined based on real-time testing feedback. After optimizing the model, it is finalized and prepared for deployment. While CNN with ADAM optimizer was used in the preferred algorithm, other machine learning methods such as Random Forest, XGBoost, etc. can be substituted to provide allergen detection and classification.

It is to be understood that a similar process may be performed for the detection of environmental allergens such as dust, pollen, mold, etc. without deterring from the inventive aspects described herein.

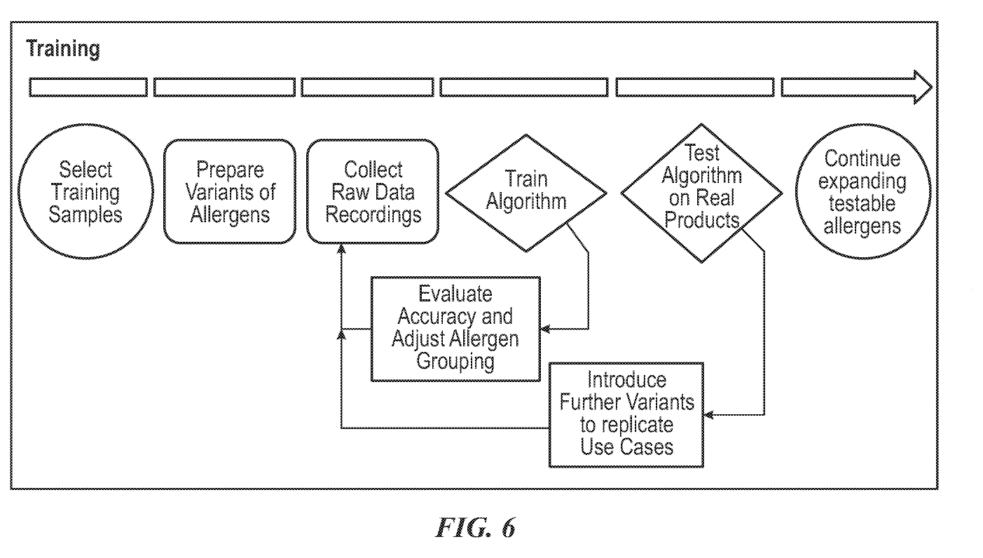

FIG. 6 illustrates a flow diagram of the model training process to improve allergen detection accuracy. Various allergen-containing foods are selected for training, ensuring diversity in the dataset. Different states of allergens (raw, roasted, processed) are considered to enhance the robustness of detection for each allergen. Next, sensor readings from allergen samples are collected at different temperature settings. Each sensor is calibrated to measure variations in conductance due to allergen exposure. Machine learning models are trained using collected data and a convolutional neural network (CNN) is used due to its ability to recognize complex signal patterns. The trained model is tested in real-world scenarios to assess effectiveness and ensure reproducibility. The accuracy of allergen classification is analyzed, and adjustments are made to improve results. Additional training data is incorporated to enhance model robustness, including different humidity and temperature environments. More allergens are gradually added to the detection system through software updates The system and method described herein can detect multiple allergens by running multiple heater profiles through the plurality of metal oxide sensors simultaneously checking for different allergens present in the sample.

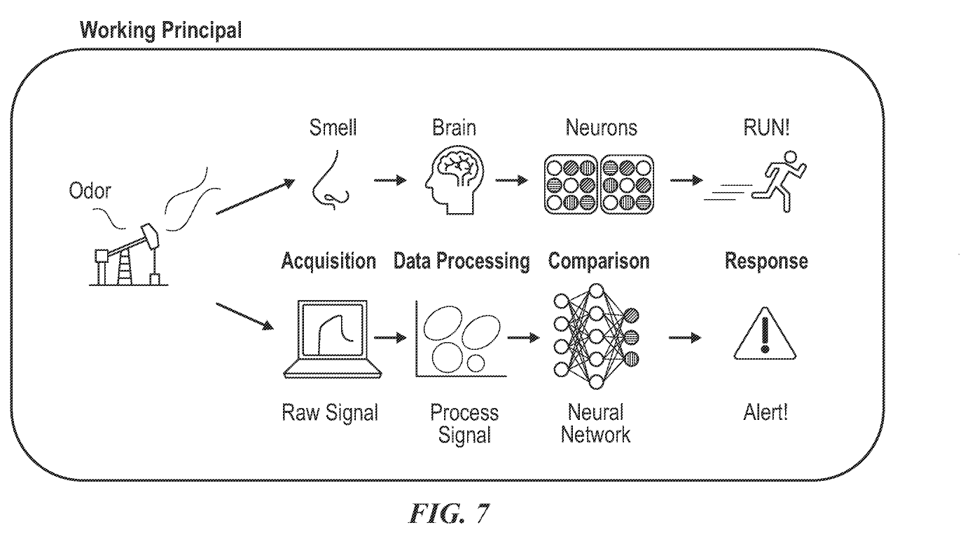

FIG. 7 illustrates a schematic of the working principle of the allergen detection system while drawing an analogy between human olfaction and machine-based allergen detection. The system mimics the human olfactory process, detecting airborne molecules from food samples. The smell sensors capture chemical signatures from the food vapors. Each sensor can be programmed with multiple heater profiles to increase sensitivity. The collected data is analyzed and transformed into a structured signal, which is then classified using trained machine learning algorithms. The processed signal is compared with trained allergen profiles using a neural network. If an allergen is detected, the system generates an alert and provides classification results. The mobile application interface can display allergen type, concentration, and detection confidence.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety to the extent allowed by applicable law and regulations. The systems and methods described herein may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

The foregoing is provided for purposes of illustrating, explaining, and describing embodiments of this disclosure. Modifications and adaptations to these embodiments will be apparent to those skilled in the art and may be made without departing from the scope or spirit of this disclosure.

As used herein and in the appended claims, the singular forms “a”, “an”, and “the” include plural referents unless the context clearly dictates otherwise.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described herein. A variety of modifications and variations are possible in light of the above teachings without departing from the following claims.

What is claimed is:

1. An allergen detection device, comprising: an olfactory sensor array arranged to be exposed to gases emitted from a food sample or an environmental sample; one or more heating elements to heat the olfactory sensor array to one or more specified temperatures; a voltage measurement device to measure a voltage signal and associate the voltage signal with a presence of one or more allergens in the food sample or the environmental sample.

2. The allergen detection device of claim 1, wherein the olfactory sensor array is comprised of one or more metal oxide sensors.

3. The allergen detection system of claim 1, wherein heating the one or more metal oxide sensors in the presence of gases causes a change in conductance of the one or more metal oxide sensors.

4. The allergen detection system of claim 3, wherein the change in conductance causes a change in the voltage signal.

5. The allergen detection device of claim 1, wherein the olfactory sensor array is attached to an auxiliary device.

6. The allergen detection device of claim 1, further comprising a transmitter to transmit the voltage signal to a display, the display to provide a metric associated with the one or more allergens.

7. The allergen detection device of claim 1, further comprising a classification device to classify the one or more allergens into one or more specific types using the voltage signal.

8. An allergen detection system, comprising: a non-contact allergen sensing device comprising: an olfactory sensor array arranged to be exposed to gases emitted from a food sample; a heating element to heat the olfactory sensor array to one or more specified temperatures; a voltmeter to measure a voltage signal received from the olfactory sensor array, wherein the voltage signal indicates a presence of one or more allergens in the food sample; a computing device to receive the voltage signal, the computing device including a memory and a processor, the memory to store operational instructions to instruct the processor to perform the following: compare the voltage signal to a plurality of references voltage signals stored in an allergen database, wherein each of the plurality of reference voltage signals corresponds to an allergen; identify the allergen present in the food sample; and display the allergen on a display of the computing device.

9. The allergen detection system of claim 8 wherein the olfactory sensor array is comprised of one or more metal oxide sensors.

10. The allergen detection system of claim 9, wherein heating the one or more metal oxide sensors in the presence of gases causes a change in conductance of the one or more metal oxide sensors.

11. The allergen detection system of claim 10 wherein the change in conductance causes a change in the voltage signal.

12. The allergen detection system of claim 11 wherein the olfactory sensor array is attached to an auxiliary device.

13. The allergen detection system of claim 12 further comprising a housing to connect to a control board, the control board in operable communication with the olfactory sensor array and the heater to enable a user to control a plurality of operational settings.

14. The allergen detection system of claim 12, further comprising a support strap connected to the control board.

15. A method for detecting allergens in a food sample, the method comprising the steps of: positioning an olfactory sensor array to be exposed to gases emitted from a food sample; heating the olfactory sensor array to one or more specified temperatures; analyzing a voltage signal received from the olfactory sensor array; referencing a database storing a plurality of variants each associated with one or more allergens to determine if the one or more allergens are present in the food sample.

16. The method of claim 15, further comprising a method for training a machine learning model, the method comprising the steps of: recording a plurality of training data; building the machine learning model to receive and interpret the plurality of training data; simulating a plurality of tests to enable the analysis of a plurality of parameters to enable refinement of the machine learning model; performing testing on the plurality of food samples.

17. The method of claim 16, wherein the olfactory sensor array is comprised of one or more metal oxide sensors, wherein heating the one or more metal oxide sensors in the presence of gases causes a change in conductance of the one or more metal oxide sensors, and wherein the change in conductance causes a change in the voltage signal.

18. The method of claim 17, wherein the olfactory sensor array is attached to an auxiliary device.

19. The method of claim 18, wherein the machine learning model is deployed remotely to a plurality of sensors.

20. The method of claim 19, wherein the machine learning model is configured to detect one or more environmental allergens.
